(19) United States
(12) Patent Application Publication
(10) Pub. No.: US 2026/0013970 A1
(43) Pub. Date: Jan. 15, 2026

(54) 2D TRACKING MARKER

(71) Applicant: Brainlab AG, Munich (DE)

(72) Inventor: Johannes Manus, Munich (DE)

(21) Appl. No.: 18/996,510

(22) PCT Filed: Sep. 30, 2022
(86) PCT No.: PCT/EP2022/077301

§ 371 Date: Jan. 17, 2025

(51) Int. Cl. A61B 90/00; A61B 34/20

(52) U.S. Cl. CPC A61B 90/39; A61B 34/20; A61B 2034/2055; A61B 2090/3983

(57) ABSTRACT

A tracking marker is provided for being positionally detected and tracked during a procedure. The tracking marker includes a substantially planar substructure having a front face and defining a plane, and a plurality of features disposed at and optically distinct from the front face, wherein the features are aligned with a grid pattern define an optical appearance which is specific for the tracking marker; and wherein at least one feature is aligned with the grid pattern and is disposed offset from the plane. A tracking marker set is provided including a plurality of tracking markers which differ from each other in their optical appearance. A computer-implemented method of identifying and positionally tracking such medical tracking markers is further provided.

### FIELD OF THE INVENTION

The present invention relates to a medical tracking marker which is configured to be positionally detected and tracked during a medical procedure, a corresponding medical tracking marker set including a plurality of such tracking markers which differ from each other in their optical appearance, as well as to a computer-implemented method of identifying and positionally tracking such medical tracking markers.

### TECHNICAL BACKGROUND

Marker tracking systems in general are used in medical technology to determine and track a position of treatment-assisting apparatus, instruments and/or parts of the patient's body. Using the positional data for the instruments, it is possible to perform medical navigation to assist physicians in treating the patient. This enables image-guided surgery to be implemented, in which physicians can see and check the position of their instruments relative to the position of treatment targets (e.g., parts of the patient's body) via a screen output, even if parts of the instruments are not longer visible. This also enables and/or greatly simplifies the planning of incisions. The data on the patient's anatomy can be obtained from previously or intra-operatively ascertained detections using imaging methods, e.g., computer tomography, nuclear-spin tomography, X-ray imaging, etc., and via a registration procedure, can be incorporated into a coordinate system of an operating theatre and/or the instrument tracking system.

While some tracking systems, for example many tracking systems that rely on a stereoscopic pair of cameras, are configured to detect spherical tracking markers, wherein the center of the two-dimensional depiction of these markers in a camera image defines the marker position, other tracking systems, which may for example rely on a monoscopic camera, are configured to determine the spatial position (i.e. the spatial location and the spatial orientation) of tracking markers and objects connected thereto by detecting complex geometric structures, for example squares, rectangles, triangles, checkerboard patterns, and analyzing how these structures appear in a two-dimensional image obtained via the monoscopic camera. However, tracking systems which focus on determining the marker position based on the detected marker center have their drawbacks in determining complex geometric structures, while tracking systems having a monoscopic camera are not suitable for detecting and tracking the spatial position of three-dimensional arrangements of (spherical) tracking markers.

It follows therefrom that known tracking systems operate in a satisfactory manner only with suitable tracking markers being provided.

The present invention has the object of providing a medical tracking marker which can be suitably used for a variety of optical tracking systems, irrespective of how camera images processed or how many cameras these tracking systems can rely on.

The present invention can be used for any procedures which involve the use of medical tracking systems such as Curve®, Kick® or Buzz®, all of them being products of Brainlab AG.

Aspects of the present invention, examples and exemplary steps and their embodiments are disclosed in the following. Different exemplary features of the invention can be combined in accordance with the invention wherever technically expedient and feasible.

### Exemplary Short Description of the Invention

In the following, a short description of the specific features of the present invention is given which shall not be understood to limit the invention only to the features or a combination of the features described in this section.

The medical tracking marker disclosed herein exhibits a generally flat design, wherein a plurality of optically detectable features are disposed at a front face thereof. Some of these features are arranged with respect to each other to define a square grid-pattern, which may be identical for a plurality of tracking markers. In order to distinguish between these tracking markers, each tracking marker exhibits an individual optical appearance which is defined by other optically detectable features which may also include features that define the grid-pattern, and which are aligned in a specific manner relative to the defined grid-pattern. At least one optically detectable feature is disposed offset from a plane that includes all of the remaining features, which renders tracking of the tracking marker more robust.

### General Description of the Invention

In this section, a description of the general features of the present invention is given for example by referring to possible embodiments of the invention.

In general, the invention reaches the aforementioned object by providing, in a first aspect, a medical tracking marker configured to be positionally detected and tracked during a medical procedure, which comprises a substantially planar substructure having a front face and defining a plane, and a plurality of features disposed at and optically distinct from the front face, wherein the plurality of features includes:
a first set of features defining, within the plane, a dimension and an orientation of a square grid pattern having an equidistant grid spacing;
a second set of features being aligned with the grid pattern and defining an optical appearance which is specific for the tracking marker; and
at least one feature being aligned with the grid pattern and being disposed offset from the plane.

In other words, the tracking marker includes one main surface which is substantially flat and exhibits several optically detectable features, wherein each one of the features serves one or more of the following purposes: A first subset of features is to define a square grid pattern having an equidistant grid spacing, i.e. a first set of equally spaced grid-lines which extend perpendicularly to a second set including the same number of equally spaced grid-lines. A second subset of features is aligned with respect to the grid pattern and is to define an individual optical appearance for a specific tracking marker, such that a distinction can be made between a plurality of tracking markers of the same type, i.e. which feature the same square grid pattern. A third subset of optically detected features which is/are also aligned with the grid pattern, is spaced from the plane containing all of the other features, such that the spatial orientation of the tracking marker can be reliably determined by a camera even if its line of sight is oriented substantially perpendicular to said plane.

In a more specific example, the tracking marker's grid pattern includes (n+2)² nodes, i.e. 4, 9, 16, etc. nodes. As the effort for determining the feature position and calculating the tracking marker's spatial position therefrom increases with the number of features to be detected, a number of 9 nodes has proven a favorable number of nodes at which optically detectable features can be disposed at.

In another example, the medical tracking marker may exhibit one or more of the following characteristics:
the dimension of the grid pattern is defined by four features defining respective corner nodes of the grid pattern;
the orientation of the grid pattern is defined by a presence or an absence of a feature at a respective side node in between two corner nodes;
the grid spacing of the grid pattern is defined by a distance between a feature disposed at a corner node and a feature disposed at a side node;
the at least one feature offset from the plane is disposed at a central node in between at least two side nodes.

In one further example, the specific optical appearance is defined by the size of features disposed at associated nodes of the grid pattern, which differs for individual features, wherein the size is selected from a limited number of predefined sizes, particularly wherein all features of the tracking marker feature either one of two predefined sizes.

As was already mentioned above, the optical appearance of the tracking marker may not only be defined by the features of the second set and the size thereof, but may additionally be defined by the features of the first set which already define the square grid pattern.

In another example, the specific optical appearance is defined by the spatial position of individual features with respect to associated nodes of the grid pattern, wherein the spatial position is selected from a limited number of predefined spatial positions, particularly wherein all features of the tracking marker feature the same size.

It becomes apparent from the examples above that the optical appearance which allows for distinguishing between different tracking markers of the same type may either be based on the difference in size of some of the features and/or on a difference in the position of some of the features with respect to the nodes of the grid pattern. While it is conceivable that only the features of the second set exhibit such deviation in size and/or position so as to define the tracking marker's optical appearance, it is also conceivable that at least some of the features of the first set exhibit a deviating size for giving the tracking marker a specific optical appearance.

In particular, the predefined spatial positions may include:
positions on one of the grid-lines adjacent to the node; and/or
positions in one of the grid-fields adjacent to the node; particularly wherein the predefined spatial positions include a predefined distance from the associated node.

In other words, the features of the second set may be spaced from their associated nodes of the grid pattern and may instead be placed at a distance therefrom and on one of the grid-lines which intersect at this node. In the alternative, the features may instead be spaced from their associated nodes and within a grid-field instead, e.g. between the grid-lines which intersect at the respective node. In particular, it is conceivable that the features are spaced from their associated nodes by a predefined distance so as to establish dedicated relative-positions between the features and their associated nodes. Further, the distance for features being spaced from their associated node may be the same for each feature of the tracking marker.

In another example, the features are disk-shaped and/or are configured to reflect incident light, and particularly
exhibit a high optical contrast towards the front face of the substructure;
include a retro-reflective coating.

Expressed in different terms, a sufficient optical difference needs to be established between the features and their surroundings, i.e. the front face of the tracking marker's substructure. This could be done by establishing a high contrast, for example disposing bright features on a dark substructure. Further, the features may have retro-reflective properties, i.e. are configured to reflect incident light back into the same spatial direction. Such features may be shaped as a disk so as to make feature detection more easy for tracking systems that derive the feature's position from the center of the feature image received by a tracking camera.

Additionally or alternatively to the above mentioned optical properties of the features, the features may include a light-emitting element, particularly an LED. In summary, any one of the features may be passive, i.e. reflect light, active, i.e. emit light, or both.

As to the at least one feature which is aligned with the grid pattern and is further disposed offset from the plane including the rest of the features, this at least one feature may be disposed in an indentation or on a projection of the front face of the tracking marker's substructure. In such case, the at least one offset feature as well as all of the remaining features can be disposed on a smooth front face of the substructure.

The present invention further relates to a medical tracking marker set which includes a plurality of tracking markers as described above. These tracking markers differ from each other in their optical appearance which is defined by the second set of features, particularly also by the first set of features, and which is specific for a respective tracking marker. Based on the optical appearance which is unique for each tracking marker, a medical tracking system is able to distinguish between the plurality of tracking markers used for a medical procedure.

In a second aspect, a computer-implemented medical method is provided which is to identify and positionally track a medical tracking marker according to one of the above described examples during a medical procedure. In one example, the method comprises the following steps:
a) first feature-set data is acquired which describes a position of the first set of features within a plane of an image obtained via an optical camera;
b) grid data is determined based on the first feature-set data, which describes a position of the grid pattern within the plane of the image;
c) second features-set data is acquired which describes a position of the second set of features within the plane of the image, particularly with respect to the first set of features;
d) identification data is determined based on the second feature-set data, particularly based on the first feature-set data as well as on the second feature-set data, which describes an identity of the tracking marker;
e) off-plane data is acquired which describes a position of the at least one feature offset from the plane, particularly with respect to the first set of features and/or with respect to the second set of features;
f) tracking data is determined based on at least one of the first feature-set data, the second feature-set data and the off-plane data, which describes a spatial position of the identified tracking marker.

It is important to note here that the method steps described above can be performed in any feasible order. For example, each one of the acquiring steps may be performed simultaneously, whereupon all of the determining steps are performed simultaneously.

In a further example, the method may involve the use of a monoscopic-camera, particularly the use of a monochrome monoscopic-camera, which is configured to optically detect the plurality of features disposed on the front face of the substructure.

In a second aspect, the invention is directed to a computer implemented medical method of identifying and positionally tracking medical tracking markers of the type described above. The invention may also relate to a computer program comprising instructions which, when the program is executed by at least one computer, causes the at least one computer to carry out the method according to the second aspect. The invention may alternatively or additionally relate to a (physical, for example electrical, for example technically generated) signal wave, for example a digital signal wave, such as an electromagnetic carrier wave carrying information which represents the program, for example the aforementioned program, which for example comprises code means which are adapted to perform any or all of the steps of the method according to the second aspect. The signal wave is in one example a data carrier signal carrying the aforementioned computer program. A computer program stored on a disc is a data file, and when the file is read out and transmitted it becomes a data stream for example in the form of a (physical, for example electrical, for example technically generated) signal. The signal can be implemented as the signal wave, for example as the electromagnetic carrier wave which is described herein. For example, the signal, for example the signal wave is constituted to be transmitted via a computer network, for example LAN, WLAN, WAN, mobile network, for example the internet. For example, the signal, for example the signal wave, is constituted to be transmitted by optic or acoustic data transmission. The invention may alternatively or additionally relate to a data stream representative of the aforementioned program, i.e. comprising the program.

In a further aspect, the invention is directed to a computer-readable storage medium on which the aforementioned program is stored. The program storage medium is for example non-transitory.

In a further aspect, the invention is directed to at least one computer (for example, a computer), comprising at least one processor (for example, a processor), wherein the program according to the second aspect is executed by the processor, or wherein the at least one computer comprises the aforementioned computer-readable storage medium.

### Definitions

In this section, definitions for specific terminology used in this disclosure are offered which also form part of the present disclosure.

The method in accordance with the invention is for example a computer-implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n-and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term computer includes a server resource. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (laaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a computer program comprising instructions which, when on the program is executed by a computer, cause the computer to carry out the method or methods, for example, the steps of the method or methods, described herein and/or to a computer-readable storage medium (for example, a non-transitory computer-readable storage medium) on which the program is stored and/or to a computer comprising said program storage medium and/or to a (physical, for example electrical, for example technically generated) signal wave, for example a digital signal wave, such as an electromagnetic carrier wave carrying information which represents the program, for example the aforementioned program, which for example comprises code means which are adapted to perform any or all of the method steps described herein. The signal wave is in one example a data carrier signal carrying the aforementioned computer program. The invention also relates to a computer comprising at least one processor and/or the aforementioned computer-readable storage medium and for example a memory, wherein the program is executed by the processor.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. A step of “determining” as described herein for example comprises or consists of issuing a command to perform the determination described herein. For example, the step comprises or consists of issuing a command to cause a computer, for example a remote computer, for example a remote server, for example in the cloud, to perform the determination. Alternatively or additionally, a step of “determination” as described herein for example comprises or consists of receiving the data resulting from the determination described herein, for example receiving the resulting data from the remote computer, for example from that remote computer which has been caused to perform the determination. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

It is the function of a marker to be detected by a marker detection device (for example, a camera or an ultrasound receiver or analytical devices such as CT or MRI devices) in such a way that its spatial position (i.e. its spatial location and/or alignment) can be ascertained. The detection device is for example part of a navigation system. The markers can be active markers. An active marker can for example emit electromagnetic radiation and/or waves which can be in the infrared, visible and/or ultraviolet spectral range. A marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is for example known to a navigation system and is for example stored in a computer of the navigation system.

In another embodiment, a marker device comprises an optical pattern, for example on a two-dimensional surface. The optical pattern might comprise a plurality of geometric shapes like circles, rectangles and/or triangles. The optical pattern can be identified in an image captured by a camera, and the position of the marker device relative to the camera can be determined from the size of the pattern in the image, the orientation of the pattern in the image and the distortion of the pattern in the image. This allows determining the relative position in up to three rotational dimensions and up to three translational dimensions from a single two-dimensional image.

The position of a marker device can be ascertained, for example by a medical navigation system. If the marker device is attached to an object, such as a bone or a medical instrument, the position of the object can be determined from the position of the marker device and the relative position between the marker device and the object. Determining this relative position is also referred to as registering the marker device and the object. The marker device or the object can be tracked, which means that the position of the marker device or the object is ascertained twice or more over time.

A marker holder is understood to mean an attaching device for an individual marker which serves to attach the marker to an instrument, a part of the body and/or a holding element of a reference star, wherein it can be attached such that it is stationary and advantageously such that it can be detached. A marker holder can for example be rod-shaped and/or cylindrical. A fastening device (such as for instance a latching mechanism) for the marker device can be provided at the end of the marker holder facing the marker and assists in placing the marker device on the marker holder in a force fit and/or positive fit.

The present invention is also directed to a navigation system for computer-assisted surgery. This navigation system preferably comprises the aforementioned computer for processing the data provided in accordance with the computer implemented method as described in any one of the embodiments described herein. The navigation system preferably comprises a detection device for detecting the position of detection points which represent the main points and auxiliary points, in order to generate detection signals and to supply the generated detection signals to the computer, such that the computer can determine the absolute main point data and absolute auxiliary point data on the basis of the detection signals received. A detection point is for example a point on the surface of the anatomical structure which is detected, for example by a pointer. In this way, the absolute point data can be provided to the computer. The navigation system also preferably comprises a user interface for receiving the calculation results from the computer (for example, the position of the main plane, the position of the auxiliary plane and/or the position of the standard plane). The user interface provides the received data to the user as information. Examples of a user interface include a display device such as a monitor, or a loudspeaker. The user interface can use any kind of indication signal (for example a visual signal, an audio signal and/or a vibration signal). One example of a display device is an augmented reality device (also referred to as augmented reality glasses) which can be used as so-called “goggles” for navigating. A specific example of such augmented reality glasses is Google Glass (a trademark of Google, Inc.). An augmented reality device can be used both to input information into the computer of the navigation system by user interaction and to display information outputted by the computer.

A navigation system, such as a surgical navigation system, is understood to mean a system which can comprise: at least one marker device; a transmitter which emits electromagnetic waves and/or radiation and/or ultrasound waves; a receiver which receives electromagnetic waves and/or radiation and/or ultrasound waves; and an electronic data processing device which is connected to the receiver and/or the transmitter, wherein the data processing device (for example, a computer) for example comprises a processor (CPU) and a working memory and advantageously an indicating device for issuing an indication signal (for example, a visual indicating device such as a monitor and/or an audio indicating device such as a loudspeaker and/or a tactile indicating device such as a vibrator) and a permanent data memory, wherein the data processing device processes navigation data forwarded to it by the receiver and can advantageously output guidance information to a user via the indicating device. The navigation data can be stored in the permanent data memory and for example compared with data stored in said memory beforehand.

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1a shows a first embodiment of the medical tracking marker;

FIG. 1b shows a perspective view of the tracking marker of FIG. 1a;

FIG. 2a shows a second embodiment of the medical tracking marker;

FIG. 2b shows a perspective view of the tracking marker of FIG. 2a;

FIG. 3 illustrates the basic steps of the method according to the second aspect.

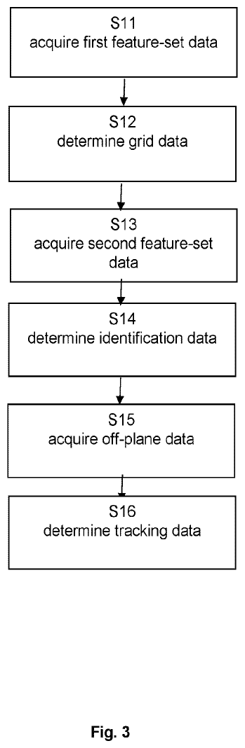

### DESCRIPTION OF EMBODIMENTS

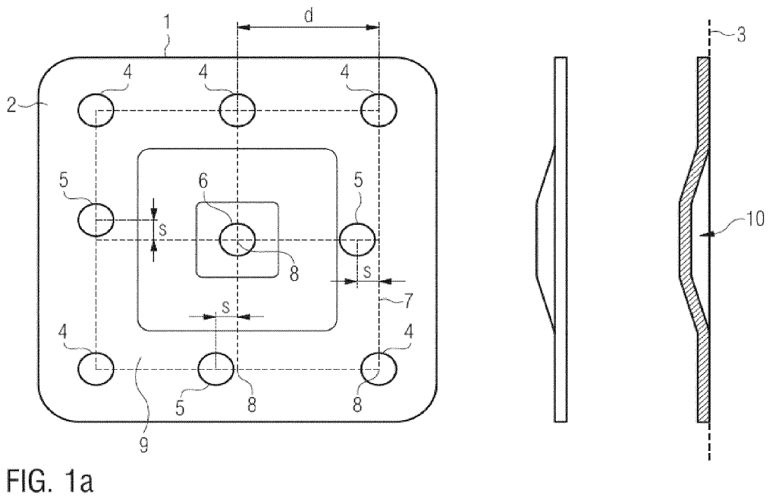

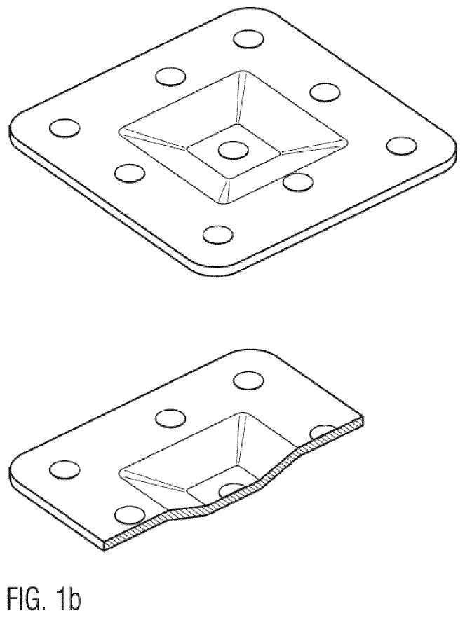

FIG. 1a shows a first embodiment of the medical tracking marker according to the first aspect. The top view on the left illustrates the arrangement of features 4, 5, 6 disposed on a front face 2 of the tracking marker's substantially planar substructure 1. Except for the central feature 6, all of the features 4, 5 are disposed within the same plane 3 defined by the front face 2, i.e. the surface of the substructure 1.

As the side-view and the cross-sectional view on the right of FIG. 1a shows, the central feature 6 is disposed in a recess 10 formed at the center of the substructure 1, and is therefore offset from plane 3 including all of the remaining features 4, 5.

A square grid pattern 7 is defined by four features 4 at each corner of the substructure 1, wherein a further feature 4 disposed at the top center of substructure 1 not only defines a distance or grid-spacing d, but also defines the “TOP”-direction of the tracking marker.

The grid pattern has a total of nine nodes 8 at which the grid-lines of the grid pattern 7 intersect and also mark out four square grid-fields 9.

In order to establish an individual optical appearance for a specific tracking marker, additional features 5 are disposed at side-nodes 8 in between the corner-nodes 8. For each one of the features 5 there is a total of three possibilities of being disposed, namely on one of a total of three grid-lines intersecting at the respective side-node 8. In the shown example, features 5 are spaced from their associated side-node 8 by a predefined distance s which is equal for every feature 5. With the possibility of disposing a total amount of three features 5 at three different positions, the shown example allows for providing 27 individual tracking markers a tracking system is able to distinguish from each other.

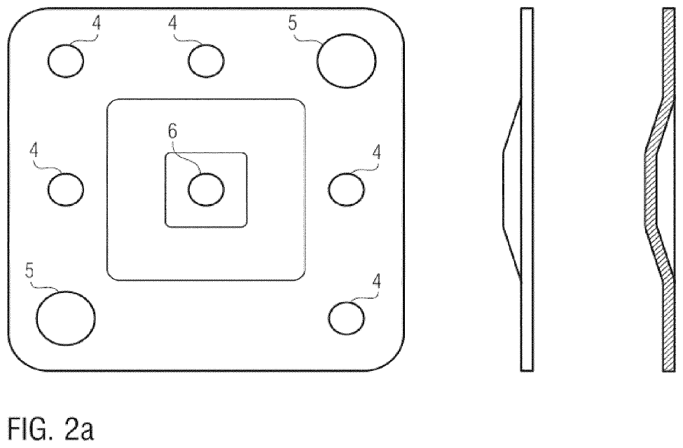

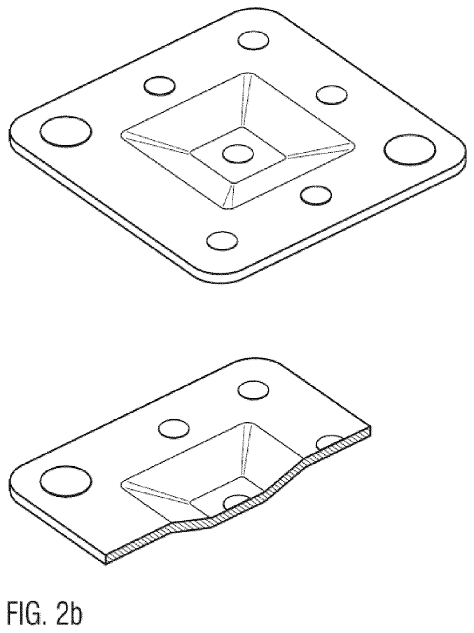

FIG. 2a shows another example of a medical tracking marker which differs from the medical tracking marker shown in FIG. 1a in that a specific optical appearance is not established by spacing some of the features 5 from their associated node 8 in a specific direction, but rather by providing features 5 having a different size as compared to the remaining features 4, 6. While all of the features 4, 5, 6 are centered right on their respective nodes 8, features 5 at the bottom left and the top right corner of the substructure 1 exhibit a larger size than the remaining features 4, 6. In this regard, it should be mentioned that features 5, amongst other features 4, define the dimension and orientation of the grid pattern 7 (cf. FIG. 1a), but also provide an optical appearance specific for one of a plurality of tracking markers.

Further, instead of centering one of the features 4 at its associated node 8 so as to define a “TOP”-direction, as explained in connection with the example of FIG. 1a, a “DOWN”-direction is defined by a missing feature at the bottom center of the substructure 1 for the example shown in FIG. 2a.

FIGS. 1b and 2b show a perspective view of the examples illustrated in FIGS. 1a and 2a, respectively.

1. A tracking marker for being positionally detected and tracked during a procedure, the tracking marker comprising:
a substantially planar substructure having a front face and defining a plane; and
a plurality of features disposed at and optically distinct from the front face,
wherein the plurality of features comprise:
a first set of features defining, within the plane, a dimension and an orientation of a square grid pattern having an equidistant grid spacing and including nine nodes;
a second set of features being aligned with the grid pattern and defining an optical appearance that is specific for the tracking marker; and
at least one feature being aligned with the grid pattern and being disposed offset from the plane,
wherein the specific optical appearance is defined by one or more of:
sizes of the plurality of features disposed at associated nodes of the grid pattern, which differ for individual features, wherein the sizes are selected from a limited number of predetermined sizes; and/or
spatial positions of the individual features relative to associated nodes of the grid pattern, wherein the spatial positions are selected from a limited set of predefined spatial positions, wherein distances for features being spaced from their associated node are the same for each feature of the tracking marker.

2. (canceled)

3. The tracking marker according to claim 1, wherein;
the dimension of the grid pattern is defined by four features defining respective corner nodes of the grid pattern;
the orientation of the grid pattern is defined by a presence or an absence of a feature at a respective side node in between two corner nodes;
the grid spacing of the grid pattern is defined by a distance between a feature disposed at a corner node and a feature disposed at a side node; and/or
the at least one feature offset from the plane is disposed at a central node in between at least two side nodes.

4. The tracking marker according to claim 1 wherein each of the features have one of two predefined sizes.

5. The tracking marker according to claim 4, wherein the optical appearance is defined by the size of features of the first set of features as well as by the size of features of the second set of features.

6. The tracking marker according to claim 1 wherein all of the features are of the same size.

7. The tracking marker according to claim 6, wherein the predefined spatial positions comprise one or more of:
positions on one of the grid-lines adjacent to the node; and/or
positions in one of the grid-fields adjacent to the node,
wherein the predefined spatial positions comprise a predefined distance(s) from the associated node.

8. The tracking marker according to claim 1, wherein the features are disk-shaped and are configured to reflect incident light,
wherein the features exhibit a high optical contrast towards the front face (2) of the substructure,
wherein the features include a retro-reflective coating.

9. The tracking marker according to claim 1, wherein each of the plurality of features comprises a light-emitting element.

10. The tracking marker according to claim 1, wherein the at least one feature offset from the plane is disposed in an indentation or on a projection of the front face of the substructure.

11. A tracking marker set comprising:
a plurality of tracking markers, wherein each of the plurality of tracking markers comprises:
a substantially planar substructure having a front face and defining a plane; and
a plurality of features disposed at and optically distinct from the front face,
wherein the plurality of features comprise:
a first set of features defining, within the plane, a dimension and an orientation of a square grid pattern having an equidistant grid spacing and including nine nodes;
a second set of features being aligned with the grid pattern and defining an optical appearance that is specific for the tracking marker; and
at least one feature being aligned with the grid pattern and being disposed offset from the plane,
wherein the specific optical appearance is defined by one or more of:
sizes of the plurality of features disposed at associated nodes of the grid pattern, which differ for individual features, wherein the sizes are selected from a limited number of predetermined sizes; and/or
spatial positions of the individual features relative to associated nodes of the grid pattern, wherein the spatial positions are selected from a limited set of predefined spatial positions, wherein distances for features being spaced from their associated node are the same for each feature of the tracking marker,
wherein the tracking markers differ from each other in the optical appearance that is defined by the second set of features, particularly by the first set of features as well as by the second set of features, and which is specific for a respective tracking marker.

12. A computer-implemented method of identifying and positionally tracking a tracking marker during a procedure, the method comprising:
acquiring first feature-set data that describes a position of a first set of features of the tracking marker within a plane of an image obtained via an optical camera;
determining grid data based on the first feature-set data, wherein the grid data describes a position of a grid pattern of the tracking marker within the plane of the image;
acquiring second features-set data that describes a position of a second set of features of the tracking marker within the plane of the image, particularly with respect to the first set of features;
determining identification data based on the first feature-set data and the second feature-set data, wherein the identification data describes an identity of the tracking marker;
acquiring off-plane data that describes a position of at least one feature offset from the plane, with respect to the first set of features and/or with respect to the second set of features; and
determining tracking data based on at least one of the first feature-set data, the second feature-set data and the off-plane data, wherein the tracking data describes a spatial position of the identified tracking marker.

13. The method according to claim 12, further comprising using a monochrome mono-camera, that is configured to optically detect and distinguish the plurality of features from a front face of the substructure.
